(19) United States
(12) Patent Application Publication
(10) Pub. No.: US 2021/0202112 A1
(43) Pub. Date: Jul. 1, 2021

(54) INTEGRATED HEALTH CONTENT FRAMEWORK

(71) Applicant: CERNER INNOVATION, INC., KANSAS CITY, KS (US)

(72) Inventors: Sameer K Bhatia, Omaha, NE (US); Gary Pilgrim, Overland Park, KS (US); Mark Winden, Leawood, KS (US)

(21) Appl. No.: 16/730,035

(22) Filed: Dec. 30, 2019

(51) Int. Cl. G16H 70/20; G16H 10/60; G06Q 50/22

(52) U.S. Cl. CPC G16H 70/20; G06Q 50/22; G16H 10/60

(57) ABSTRACT

Embodiments herein disclose systems, methods, and computer-readable media for providing relevant content to users. Content from a plurality of disparate sources can be received and data from an electronic health record (EHR) can be leveraged such that relevant data or recommendations can be provided to users.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims as supported by the Specification, including the Detailed Description and Drawings.

In brief and at a high level, embodiments of the present invention provide systems, methods, and computer-readable media for providing contextually relevant health information. Embodiments provide an application and/or cloud-based service that provide contextually relevant health information based on disease state. The information can be provided to a health care provider, a patient, or a combination thereof. The information can be relevant to a specific patient, a patient population, and the like.

One embodiment provides one or more non-transitory computer-readable media having computer-executable instructions embodied thereon that, when executed by a processor of a computer device, perform a method. The method comprises receiving a plurality of protocols for a plurality of clinical conditions from one or more protocol sources; receiving, from one or more electronic health record (EHR) sources, EHR data; applying the plurality of protocols to the health data from the one or more EHR sources; identifying at least a first protocol that conflicts with at least a portion of the EHR data; identifying at least a second protocol that is consistent with the EHR data; and generating a recommendation for an action to take that is consistent with the second protocol.

Another embodiment provides a computerized method, the method comprising receiving a plurality of protocols for a plurality of clinical conditions from one or more protocol sources; receiving, from one or more electronic health record (EHR) sources, EHR data; applying the plurality of protocols to the health data from the one or more EHR sources; identifying at least a first protocol that conflicts with at least a portion of the EHR data; identifying at least a second protocol that is consistent with the EHR data; and generating a recommendation for an action to take that is consistent with the second protocol.

Yet another embodiment provides a system. The system comprises one or more processors configured to receive a plurality of protocols for a plurality of clinical conditions from one or more protocol sources; receive, from one or more electronic health record (EHR) sources, EHR data; apply the plurality of protocols to the health data from the one or more EHR sources; identify at least a first protocol that conflicts with at least a portion of the EHR data; identify at least a second protocol that is consistent with the EHR data; and generate a recommendation for an action to take that is consistent with the second protocol.

### BRIEF DESCRIPTION OF THE DRAWINGS

Embodiments are described in detail below with reference to the attached drawings figures, wherein:

FIG. 1 depicts a block diagram of an exemplary system architecture in accordance with an embodiment of the present invention;

FIG. 2 depicts a block diagram of an exemplary manager in accordance with an embodiment of the present invention;

FIG. 3 is a flow diagram of an exemplary method in accordance with an embodiment of the present invention; and

FIG. 4 depicts a block diagram of an exemplary computing environment suitable to implement embodiments of the present invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

As one skilled in the art will appreciate, embodiments of the disclosure may be embodied as, among other things: a method, system, or set of instructions embodied on one or more computer-readable media. Accordingly, the embodiments may take the form of a hardware embodiment, a software embodiment, or an embodiment combining software and hardware. In one embodiment, the invention takes the form of a computer-program product that includes computer-useable instructions embodied on one or more computer-readable media, as discussed further herein.

Embodiments of the present invention provide systems, methods, and computer-readable media for providing contextually relevant health information. Embodiments provide contextually relevant health information to patients, health care providers, and the like, using a combination of data from one or more electronic health records (EHRs) and one or more treatment protocols. In particular, the one or more treatment protocols can be applied to the EHR data to identify a protocol that should be used, according to the specific EHR data. Additional data from external sources can also be applied to the EHR data including environmental data, demographic data, location data, and the like.

At a high level, embodiments of the present invention provide contextually relevant health information. The software product can communicate with one or more disparate sources to, among other things, access data from an EHR system, store health data in an EHR system, generate one or more recommendations, and the like. The software product can further communicate with additional disparate sources such as third-party content providers including weather content providers, location content providers, fitness data content providers, and the like. The software product can further communicate with disparate content sources that provide one or more protocols for disease states. A protocol, as used herein, refers to an adopted standard of care for a specific disease state. For example, many expert entities provide treatment protocols for specific disease states (e.g., MD Anderson Cancer center provides treatment protocols for a variety of cancers). These protocols are typically an “if/then”-type decision tree and are not used in combination with EHR data to provide recommendations specific to the EHR data and one or more additional content items (e.g., weather data, fitness data, location data, etc.). In other words, the data from these disparate sources would be beneficial if integrated into the EHR.

The present solution seeks to solve this technical problem by providing a central hub that is integrated with both the EHR system and one or more disparate sources that provide protocol data while also being capable of integration with any number of additional disparate sources that provide additional content such as weather, location, fitness habits, diet habits, financial information, and the like.

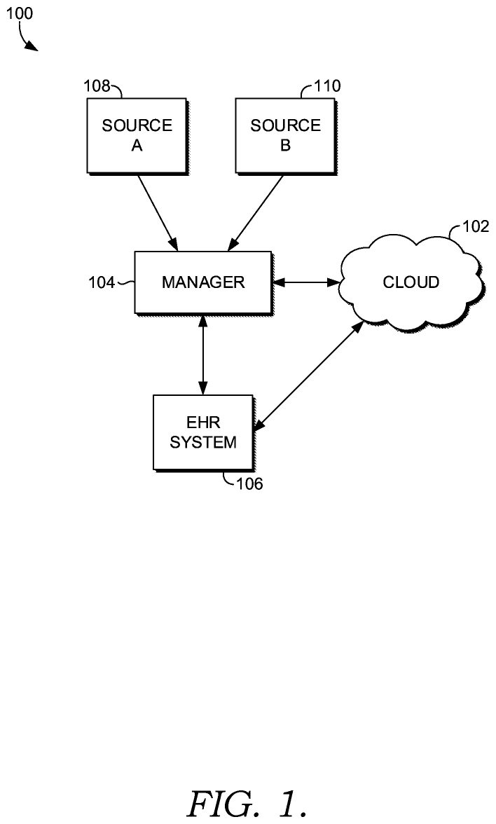

Referring to the drawings in general, and initially to FIG. 1, a block diagram illustrating an exemplary system 100 architecture in which some embodiments of the present disclosure may be employed. It should be understood that this and other arrangements described herein are set forth only as examples. Other arrangements and elements (e.g., machines, interfaces, functions, orders, and groupings of functions, etc.) can be used in addition to or instead of those shown, and some elements may be omitted altogether. Further, many of the elements described herein are functional entities that may be implemented as discrete or distributed components or in conjunction with other components, and in any suitable combination and location. Various functions described herein as being performed by one or more entities may be carried out by hardware, firmware, and/or software. For instance, various functions may be carried out by a processor executing instructions stored in memory.

It should be understood that the system 100 shown in FIG. 1 is an example of one suitable computing system architecture. Each of the components of FIG. 1 may be implemented via any type of computing device. The components can communicate with each other via a network including, without limitation, one or more local area networks (LANs) and/or wide area networks (WANs). Such networking environments are commonplace in offices, enterprise-wide computer networks, intranets, and the Internet. It should be understood that any number of components shown in FIG. 1 may be employed within the system 100 within the scope of the present invention. Each may be implemented via a single device or multiple devices cooperating in a distributed environment. Additionally, other components not shown may also be included within the environment.

Among other components not shown, the system 100 includes a manager 104, an EHR system 106, and one or more disparate sources 108 and 110, any of which can interact with any other component of the system 100 and each of which are communicatively coupled with each other. These components may communicate with each other via networking means (e.g., network 102) which may include, without limitation, one or more local area networks LANs and/or wide area networks (WANs). In exemplary implementations, such networks comprise the Internet and/or cellular networks, amongst any of a variety of possible public and/or private networks.

A source device (not shown) can comprise any type of computing device capable of use by a user and capable of providing any of the content discussed herein. By way of example and not limitation, a source device can be embodied as a personal computer (PC), a laptop computer, a mobile device, a smartphone, a tablet computer, a smart watch, a wearable computer, a fitness tracker, a personal digital assistant (PDA) device, a global positioning system (GPS) device, a video player, a handheld communications device, an embedded system controller, a camera, a remote control, a wearable electronic device with a camera (e.g., smart glasses, gesture-based wearable computers, etc.) a consumer electronic device, a workstation, or any combination of these delineated devices, a combination of these devices, or any other suitable computer device. The source device, as applied herein, can be utilized to, for instance, access the EHR system to receive recommendations of the system 100, supply protocols, supply external data such as demographic or environmental data, and the like.

The EHR system 106 can be any system that maintains, and provides access to, one or more EHR database(s) containing records of treatment events, medication history, diagnoses, problems, allergies, demographic attributes, laboratory tests, time and date data, and any other health-related data, or any combination thereof for a plurality of patients and/or patient populations. Additionally, the EHR system 106 can include clinical notes, appointment notes, records of issued prescriptions, diagnoses, care plans, bloodwork, urinalysis, treatment data, emergency contact information, and the like, for each patient of a healthcare facility or a plurality of healthcare facilities. Further, EHR system 106 can include images, representations, or clinical documentation of physical health data (e.g., X-rays, CT scans, ultrasound images, etc.). Additionally, in some embodiments, EHR system 106 can maintain one or more pharmaceutical formularies that identify prescriptions prescribed by, or available for prescription by, care providers.

The one or more disparate sources, shown as Source A 108 and Source B 110, can be any source system or device that provides any content utilized by the manager 104. Exemplary sources, as previously mentioned, can be sources that provide any content relevant to an individual or a population including, but not limited to, protocol standards, weather data, fitness data, location data, air quality data, traffic data, accident data, natural disaster data, and the like.

The manager 104 can be any device that can integrate with an EHR system (such as EHR system 106) and one or more disparate sources and apply the data/content received from the one or more disparate sources to the data from the EHR system to generate personalized recommendations relevant to the EHR data of a specific patient and/or a specific patient population.

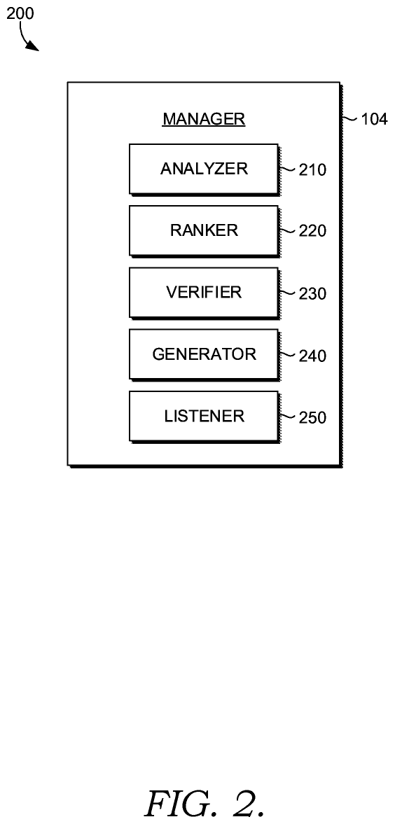

Having briefly described the components of system 100, exemplary component interactions of the components of FIGS. 1 and 2 are now described. The manager 104 can include an analyzer 210, a ranker 220, a verifier 230, a generator 240, and a listener 250. While shown separately, any of the described components can be combined into a single component.

The analyzer 210 can evaluate a variety of data from disparate sources. In embodiments, the analyzer 210 receives one or more protocols from one or more protocol sources. Protocol sources can be experts in a field such that the protocols associated therewith are deemed the standard of care for a specific disease state. The analyzer 210 can also receive health data from an EHR server. The health data can be specific to a patient (e.g., a patient's EHR) or specific to an entire patient population (e.g., individuals under the age of 18 with asthma).

Additionally, the analyzer 210 can receive data from one or more additional sources that are disparate from the EHR system. The additional sources can be sources that provide information related to environmental data, demographic data, or any other data that can be applied to health data. By way of example, and not limitation, the additional data can be weather information (e.g., high pollen count), fitness information (e.g., a patient just ran 1.3 miles), air quality information (e.g., air quality is low), natural disaster information (e.g., forest fire is near), location information (e.g., proximity of entities to an individual such as pharmacies, fitness centers, outpatient facilities, etc.), a history (e.g., patient had asthma attack two weeks ago, patient is obese, etc.), financial information (e.g., patient has no insurance), and the like.

The analyzer 210 can evaluate the received data (e.g., EHR data, protocol data, environmental data, demographic data, etc.) in view of the EHR data. In particular, the analyzer 210 can identify one or more criteria within EHR data and leverage that data against the protocols, for instance, to identify a protocol that is best for a patient. Upon identifying criteria that are inconsistent with a protocol (or vice versa), a protocol is deemed by the analyzer 210 to not be the preferred protocol for the patient (or patient population, depending on the analysis). In the alternative, upon identifying criteria that is consistent with a protocol, the protocol can be deemed as a preferred protocol. Exemplary criteria can include age, sex, rage, allergies, problems, insurance information, disease state, and the like.

Once identified as being consistent with EHR data or, rather, not identified as being inconsistent with EHR data, a protocol can be verified by the verifier 230. The verifier 230 can further evaluate one or more protocols to identify if a predetermined number of criteria are consistent with the protocols before identifying the protocol as the preferred protocol. Thus, in embodiments, a protocol is consistent with EHR data but not the preferred protocol.

In the event more than one protocol is identified as having a predetermined number of criteria consistent with the EHR data, a ranker 220 can rank the protocols and the ranking can be provided by the system. Rankings can be provided based on configurable criteria including a date of a protocol, an expert level ranking of the source of the protocol, citations of the protocol in literature, and the like. The ranker 220 can populate a ranking score and associate the ranking score with an associated protocol.

The analyzer 210 can further apply data in addition to the EHR data. For example, protocols or recommended actions can be associated with a cost, an outcome, and the like. The analyzer 210 can identify a recommended action based on any one of, or a combination of, factors. For instance, a drug that is less expensive and has better outcomes may be recommended over a different drug listed in a protocol. By way of further example, the analyzer 210 can apply external data to one or more of the EHR data and the protocol(s) to identify a recommended protocol and/or a recommendation action for a patient/patient population.

The manager 104 can further comprise a generator 240. Generator 240 can generate a recommendation for an action to take that is consistent with a preferred protocol. In embodiments, the recommendation is unrelated to the preferred protocol. In additional embodiments, the recommendation is directly from the preferred protocol. The recommendation can be provided in the form of an alert and can be provided to health care providers, patients, health care entities, combinations thereof, and the like. The recommendation can be provided on any device capable of providing recommendations in a visual format, audio format, or a combination thereof. The recommendation can be provided on a display of a device.

The manager can further comprise a listener 250. Listener 250 can “listen” for changes to any of the content in order to dynamically update recommendations. For instance, if information is entered into a patient's EHR, the information may conflict with a recommendation and, thus, the recommendation may need to be updated or a protocol can be modified such that it is no longer consistent with specific EHR data and, thus, cannot be recommended as a preferred protocol, for example. In those situations, the listener 250 can identify any change in content and notify the analyzer 210. The analyzer 210 can initiate the process, beginning with an evaluation of the content available. Alternatively, the listener 250 can identify the change in the content and notify the analyzer 210 of the change along with a notification of whether the change requires analysis. For example, if the listener 250 detects a change in a patient's address but it does not affect the recommendation for the patient to consult with a nutritionist, further analysis is not needed and the listener 250 could identify that no analysis is needed. Rather, if the listener 250 detects a confirmed visit with a nutritionist, the listener 250 could identify that the recommendation is no longer needed and notify the analyzer 210 to remove the recommendation as it has been completed. The analyzer 210, in that instance, can remove the recommendation directly from the EHR. In alternative embodiments, the analyzer 210 can provide a notification to a user to remove the recommendation or approve removal of the recommendation from the EHR.

In embodiments, the recommendation is a proactive recommendation. As used herein, a proactive recommendation is a recommendation that is identified by the system based on a probability above a certain threshold of a certain result occurring. For instance, air quality indicators can be an indication of asthma attacks. Thus, the system 100 can generate a recommendation to a patient with asthma, an asthma patient population, an asthma healthcare provider, an asthma treatment facility, and the like, to take proactive measures to avoid an asthma attack (e.g., take X drug 2× daily instead of one time daily for X number of days), as an attack is highly likely based on current conditions.

The system 100 described herein can further utilize artificial intelligence capabilities to notify a user when a selection should be evaluated, for instance. For example, if a user selects recommendation A for treatment of infection but should have selected recommendation B based on any number of factors, the system 100 can notify the user that recommendation B should have been selected. For instance, a patient may have a preference noted that is a contraindication of recommendation A such that recommendation B should be selected.

Alternatively, if a user continues to select recommendations that are not initially recommended by the system 100, machine learning capabilities can be utilized to refine the recommendations provided. For instance, if a user typically selects one protocol over another in a specific situation or for a specific patient/population, despite the selected protocol not being identified as the preferred protocol by the system, the system 100 can learn that pattern and note the typical selection to the user. In the example above regarding infection, if the user always selects recommendation A, even though the system 100 recommends recommendation B, the system 100 can identify recommendation B as a recommendation specific to the user.

In additional embodiments, recommendations can be ranked as a high/medium/low priority such that the system 100 can automatically manage recommendations of a certain priority without user input. For example, a recommendation that is associated with a low risk can be modified, updated, replaced, removed by the system 100 when certain factors are identified to warrant additional action (e.g., a recommendation is completed so it can be removed, a protocol has changed so the recommendation needs to be updated or replaced, etc.). Conversely, a recommendation that is associated with a medium or high priority level will require user action prior to any changes to the recommendation by the system. Any other ranking priority can be utilized such as a numerical scale (1-10) or the like.

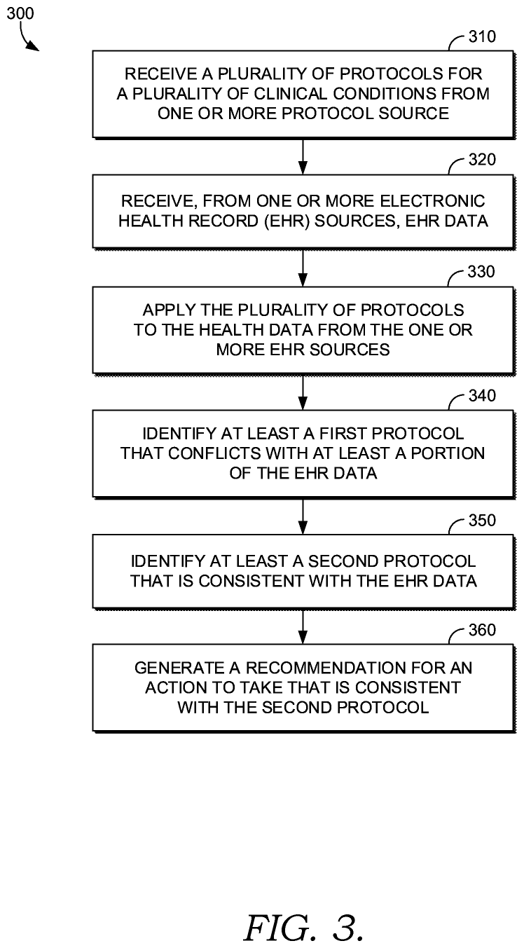

Turning now to FIG. 3, a flow diagram is provided showing a method 300 in accordance with some embodiments of the present invention. Initially, at block 310, a plurality of protocols for a plurality of clinical conditions from one or more protocol sources is received. At block 320, EHR data is received from one or more electronic health record (EHR) sources. The plurality of protocols to the health data from the one or more EHR sources at block 330. At least a first protocol that conflicts with at least a portion of the EHR data is identified at block 340. At block 350, at least a second protocol that is consistent with the EHR data is identified. At block 360, a recommendation for an action to take that is consistent with the second protocol is generated.

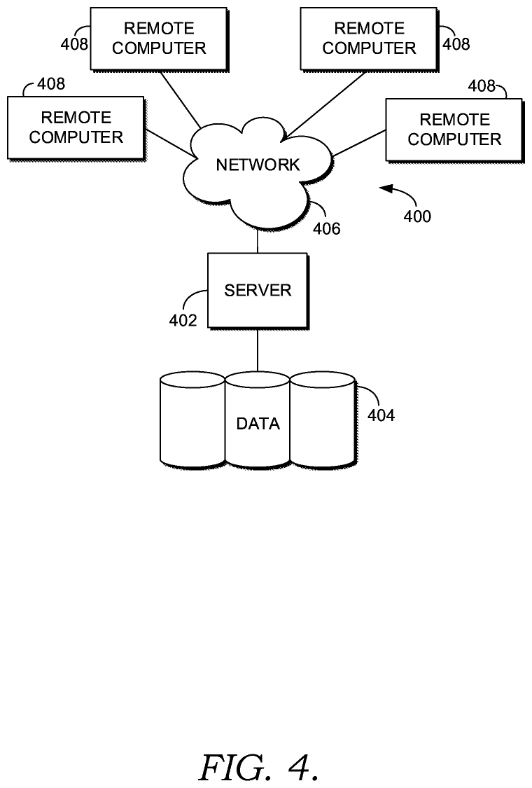

Turning to FIG. 4, it depicts a block diagram of an exemplary environment suitable to implement embodiments of the present invention. The exemplary computing environment 500 is suitable to implement embodiments of the present invention. It will be understood by those of ordinary skill in the art that the exemplary computing environment 400 is just one example of a suitable computing environment and is not intended to limit the scope of use or functionality of the present invention. Similarly, the exemplary computing environment 400 should not be interpreted as imputing any dependency and/or any requirements with regard to each component and combination(s) of components illustrated in FIG. 4. It will be appreciated by those having ordinary skill in the art that the connections illustrated in FIG. 4 are also exemplary as other methods, hardware, software, and devices for establishing a communications link between the components, devices, systems, and entities, as shown in FIG. 4, may be utilized in implementation of the present invention. Although the connections are depicted using one or more solid lines, it will be understood by those having ordinary skill in the art that the exemplary connections of FIG. 4 may be hardwired or wireless, and may use intermediary components that have been omitted or not included in FIG. 4 for simplicity's sake. As such, the absence of components from FIG. 4 should be not be interpreted as limiting the present invention to exclude additional components and combination(s) of components. Moreover, though devices and components are represented in FIG. 4 as singular devices and components, it will be appreciated that some embodiments may include a plurality of the devices and components such that FIG. 5 should not be considered as limiting the number of a devices or components.

Continuing, the exemplary computing environment 400 of FIG. 4 is illustrated as being a distributed environment where components and devices may be remote from one another and may perform separate tasks. The components and devices may communicate with one another and may be linked to each other using a network 406. The network 406 may include wireless and/or physical (e.g., hardwired) connections. Exemplary networks include a telecommunications network of a service provider or carrier, Wide Area Network (WAN), a Local Area Network (LAN), a Wireless Local Area Network (WLAN), a cellular telecommunications network, a Wi-Fi network, a short range wireless network, a Wireless Metropolitan Area Network (WMAN), a Bluetooth® capable network, a fiber optic network, or a combination thereof. The network 406, generally, provides the components and devices access to the Internet and web-based applications. The exemplary environment may also be a cloud computing environment.

The exemplary computing environment 400 comprises a computing device in the form of a server 402. Although illustrated as one component in FIG. 4, the present invention may utilize a plurality of local servers and/or remote servers in the exemplary computing environment 400. The server 402 may include components such as a processing unit, internal system memory, and a suitable system bus for coupling to various components, including a database or database cluster. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

The server 402 may include or may have access to computer-readable media. Computer-readable media can be any available media that may be accessed by server 402, and includes volatile and nonvolatile media, as well as removable and non-removable media. By way of example, and not limitation, computer-readable media may include computer storage media and communication media. Computer storage media may include, without limitation, volatile and nonvolatile media, as well as removable and non-removable media, implemented in any method or technology for storage of information, such as computer-readable instructions, data structures, program modules, or other data. In this regard, computer storage media may include, but is not limited to, Random Access Memory (RAM), Read-Only Memory (ROM), Electrically Erasable Programmable Read-Only Memory (EEPROM), flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the server 402. Computer storage media does not comprise signals per se.

Communication media typically embodies computer-readable instructions, data structures, program modules, or other data in a modulated data signal, such as a carrier wave or other transport mechanism, and may include any information delivery media. As used herein, the term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, radio frequency (RF), infrared, and other wireless media. Combinations of any of the above also may be included within the scope of computer-readable media.

In embodiments, the server 402 uses logical connections to communicate with one or more remote computers 408 within the exemplary computing environment 400. In one embodiment, the one or more remote computers 408 comprise external computer systems that leverage object-oriented programming. In embodiments where the network 406 includes a wireless network, the server 402 may employ a modem to establish communications with the Internet, the server 402 may connect to the Internet using Wi-Fi or wireless access points, or the server 402 may use a wireless network adapter to access the Internet. The server 402 engages in two-way communication with any or all of the components and devices illustrated in FIG. 4, using the network 406. Accordingly, the server 402 may send data to and receive data from the remote computers 408 over the network 406.

Although illustrated as a single device, the remote computers 408 may include multiple computing devices. In an embodiment having a distributed network, the remote computers 408 may be located at one or more different geographic locations. In an embodiment where the remote computers 408 are a plurality of computing devices, each of the plurality of computing devices may be located across various locations such as buildings in a campus, medical and research facilities at a medical complex, offices or “branches” of a banking/credit entity, or may be mobile devices that are wearable or carried by personnel, or attached to vehicles or trackable items in a warehouse, for example.

In some embodiments, the remote computers 408 are physically located in a medical setting such as, for example, a laboratory, inpatient room, an outpatient room, a hospital, a medical vehicle, a veterinary environment, an ambulatory setting, a medical billing office, a financial or administrative office, hospital administration setting, an in-home medical care environment, and/or medical professionals' offices. By way of example, a medical professional may include physicians; medical specialists such as surgeons, radiologists, cardiologists, and oncologists; emergency medical technicians; physicians' assistants; nurse practitioners; nurses; nurses' aides; pharmacists; dieticians; microbiologists; laboratory experts; genetic counselors; researchers; students; and the like. In other embodiments, the remote computers 408 may be physically located in a non-medical setting, such as a packing and shipping facility or deployed within a fleet of delivery or courier vehicles. Remote computers 408 can also be hosted on a private or public cloud.

Continuing, the exemplary computing environment 400 includes a database 404. In some embodiments, the database 404 and at least the server 402, together, form a relational database management system. Although shown as a single component, the database 404 may be implemented using multiple data stores that are communicatively coupled to one another, independent of the geographic or physical location of a memory device. Exemplary data stores may also store data in the form of electronic records, for example, electronic medical records of patients, transaction records, billing records, task and workflow records, chronological event records, and the like. Database 404 can also be hosted on a private or public cloud.

Generally, the database 404 includes physical memory that is configured to store information encoded in data. For example, the database 404 may provide storage for computer-readable instructions, computer-executable instructions, data structures, data arrays, computer programs, applications, and other data that supports the functions and action to be undertaken using the exemplary computing environment 400 and components shown in exemplary FIG. 4.

In a computing environment having distributed components that are communicatively coupled via the network 406, program modules may be located in local and/or remote computer storage media including, for example only, memory storage devices. Embodiments of the present invention may be described in the context of computer-executable instructions, such as program modules, being executed by a computing device. Program modules may include, but are not limited to, routines, programs, objects, components, and data structures that perform particular tasks or implement particular data types. In embodiments, the server 402 may access, retrieve, communicate, receive, and update information stored in the database 404, including program modules. Accordingly, the server 402 may execute, using a processor, computer instructions stored in the database 404 in order to perform embodiments described herein.

Although internal components of the devices in FIG. 4, such as the server 402, are not illustrated, those of ordinary skill in the art will appreciate that internal components and their interconnection are present in the devices of FIG. 4. Accordingly, additional details concerning the internal construction of the device are not further disclosed herein.

The present invention has been described in relation to particular embodiments, which are intended in all respects to be illustrative rather than restrictive. Further, the present invention is not limited to these embodiments, but variations and modifications may be made without departing from the scope of the present invention.

From the foregoing, it will be seen that this invention is one well adapted to attain all the ends and objects set forth above, together with other advantages which are obvious and inherent to the system and method. It will be understood that certain features and subcombinations are of utility and may be employed without reference to other features and subcombinations.

What is claimed is:

1. One or more non-transitory computer-readable media having executable instructions embodied thereon that, when executed by a processor of a computer device, perform a method, the method comprising:
receiving a plurality of protocols for a plurality of clinical conditions from one or more protocol sources;
receiving, from one or more electronic health record (EHR) sources, EHR data;
applying the plurality of protocols to the health data from the one or more EHR sources;
identifying at least a first protocol that conflicts with at least a portion of the EHR data;
identifying at least a second protocol that is consistent with the EHR data; and
generating a recommendation for an action to take that is consistent with the second protocol.

2. The media of claim 1, wherein the further comprising providing the recommendation to a health care provider.

3. The media of claim 1, further comprising providing the recommendation to a patient associated with the EHR data.

4. The media of claim 1, wherein the plurality of protocols and the EHR data are received at a cloud-based location.

5. The media of claim 1, further comprising verifying the recommendation by identifying a number of criteria within the EHR data that is consistent with the second protocol, wherein the number is greater than a predetermined threshold.

6. The media of claim 1, further comprising providing the recommendation along with a ranked list of alternate recommendations.

7. The media of claim 6, wherein at least one recommendation within the ranked list of alternate recommendations is from the first protocol and is ranked lower than the recommendation.

8. The media of claim 1, further comprising receiving additional data from one or more external sources associated with one or more of environmental information or demographical information of either a patient or a patient population.

9. The media of claim 1, wherein the recommendation is specific to a patient.

10. The media of claim 1, wherein the recommendation is specific to a patient population.

11. A computerized method, the method comprising:
receiving a plurality of protocols for a plurality of clinical conditions from one or more protocol sources;
receiving, from one or more electronic health record (EHR) sources, EHR data;
applying the plurality of protocols to the health data from the one or more EHR sources;
identifying at least a first protocol that conflicts with at least a portion of the EHR data;
identifying at least a second protocol that is consistent with the EHR data; and
generating a recommendation for an action to take that is consistent with the second protocol.

12. The method of claim 11, further comprising receiving additional data from one or more external sources associated with one or more of environmental information or demographical information.

13. The media of claim 11, wherein the recommendation is specific to one or more of a patient or a patient population.

14. The media of claim 11, further comprising verifying the recommendation by identifying a number of criteria within the EHR data that is consistent with the second protocol, wherein the number is greater than a predetermined threshold.

15. The media of claim 11, further comprising providing the recommendation to one or more of a health care provider and a patient associated with the EHR data.

16. A system for providing contextually relevant content, the system comprising:
one or more processors to:
receive a plurality of protocols for a plurality of clinical conditions from one or more protocol sources;
receive, from one or more electronic health record (EHR) sources, EHR data;
apply the plurality of protocols to the health data from the one or more EHR sources;
identify at least a first protocol that conflicts with at least a portion of the EHR data;
identify at least a second protocol that is consistent with the EHR data; and
generate a recommendation for an action to take that is consistent with the second protocol.

17. The system of claim 16, wherein the one or more processors are further configured to provide the recommendation to one or more of a health care provider and a patient associated with the EHR data.

18. The system of claim 16, wherein the recommendation is specific to one or more of a patient or a patient population.

19. The system of claim 16, wherein the one or more processors are further configured to receive additional data from one or more external sources associated with one or more of environmental information or demographical information.

20. The system of claim 16, wherein the one or more processors are further configured to provide the recommendation along with a ranked list of alternate recommendations.
